(19) United States
(12) Patent Application Publication
(10) Pub. No.: US 2011/0144515 A1
(43) Pub. Date: Jun. 16, 2011

(54) SYSTEMS AND METHODS FOR PROVIDING ENVIRONMENT MONITORING

(75) Inventors: Charlene Warres Bayer, Marietta, GA (US); Robert James Hendry, Powder Springs, GA (US); Mark Lehi Jones, Atlanta, GA (US)

(73) Assignee: Georgia Tech Research Corporation, Atlanta, GA (US)

(21) Appl. No.: 12/867,573

(22) PCT Filed: Feb. 16, 2009
(86) PCT No.: PCT/US2009/034210

§ 371 Date: Feb. 22, 2011

Related U.S. Application Data
(60) Provisional application No. 61/028,939, filed on Feb. 15, 2008.

(51) Int. Cl. A61B 5/08

(52) U.S. Cl. 600/529

(57) ABSTRACT

The present invention describes systems and methods for providing portable environment monitoring systems. An exemplary embodiment of the present invention provides a portable environment monitoring system comprising a sensor enabled to sense an airborne analyte. The portable environment monitoring system also includes a microprocessor in communication with the sensor and enabled to process information received from the sensor. Additionally, the portable environment monitoring system includes a memory device in communication with the microprocessor and enabled to store information received from the microprocessor. Furthermore, a user is enabled to ambulate with the portable environment monitoring system.

### CROSS REFERENCE TO RELATED APPLICATION

This application claims the benefit of U.S. Provisional Patent Application No. 61/028,939, filed 15 Feb. 2008, the entire contents and substance of which are hereby incorporated by reference as if fully set forth below.

### STATEMENT REGARDING FEDERALLY SPONSORED RESEARCH OR DEVELOPMENT

This invention was made with United States Government support in the Healthy Homes and Lead Hazard Control Grant Program sponsored by the United States Department of Housing and Urban Development under the Agreement No. GALHH0124-04. The Government has certain rights in this Invention.

### FIELD OF THE INVENTION

The present invention relates generally to systems and methods for providing an environment monitoring system and, more particularly, to systems and methods for providing portable environment monitoring systems.

### BACKGROUND OF THE INVENTION

Despite better medical treatment and overall access to medical care, asthma is a growing and significant health problem throughout the nation and the world, particularly among children. Asthma, the most chronic respiratory disorder in the U.S. population, affects approximately 17.3 million Americans including over 5 million children. Long-term surveillance data show that both the prevalence and morbidity of asthma in the U.S. are on the rise. Children account for a large portion of this increase. From 1980 to 1994, asthma case rates in children age 0-4 years increased by 160%. The bar graphs provided in FIG. 1 illustrate the percentages of asthma case rates in categories of age, race, and gender. As clearly demonstrated by FIG. 1, adolescents comprise a majority of the number of asthma cases.

It is estimated that asthma annually costs the US $14 billion. Sixteen percent of children in low-income families compared to 11% in higher economic families were more likely to have asthma. The cost of the growing worldwide asthma epidemic is not only in dollars. Annually in the US, asthma is responsible for 500,000 hospitalizations (214,000 involve children), 4,500 deaths, 14 million missed school days, 14.5 million missed work days, and 134 million days of restricted activity. The overall number of people with asthma in the US has increased by 102% between 1979-1980 and 1993-1994. Asthma is a potentially life-threatening disease. It can be debilitating for the patients, limiting their activities for work and leisure. Many in the medical community believe that we are causing the increase in asthma, particularly among children, by not adequately controlling pollution in our environment.

Although many in the field assume that environmental exposures have direct causal links to asthma and that indoor and outdoor environmental contaminants play an important role in the inception of asthma early in life and later as triggers for asthma exacerbations, to date these links have not been established due to the absence of instrumentation that can measure a matrix of airborne contaminant concentrations and concurrently measure lung function. Demonstrating these links, as well as identifying the actual triggers, is critical since the average child spends 80-90% of their time indoors, increasing their risks from exposure to indoor pollutants, and indoor airborne pollution levels may be as much as ten to hundreds of times higher than outdoors; and also it is now recognized that outdoor air pollutants, particularly ozone and particles, can penetrate the building shell and enter the indoor environment.

The Federal government recognizes the importance of pediatric asthma to the overall quality of life in the US. Executive Order 13045 issued by President Clinton established a Task Force charged with developing a plan to promote federal action and strategies to protect all children with asthma from environmental risks that worsen asthma. NIH has designated a day in May each year as Asthma Alert Day. The Federal government's vision of Healthy People 2010 lists Environmental Health and Respiratory Diseases as major areas of concern. Several Federal agencies have major research programs and centers investigating the causes, treatment, and control of asthma. Yet even with all of this ongoing research, there is still no clear link to the relationships between asthma, and other chronic diseases, and environmental exposures.

A confounding issue in characterizing adverse health effects resulting from air pollution exposure is that polluted air is a complex mixture of volatile gases, both organic and inorganic, suspended particles of a wide range of sizes, bioaerosols, and other irritating compounds. The complexity of this mixture presents a difficult challenge to relate specific health effects to specific pollutants, and in reality, the composition of the mixture may be more important than the individual components.

This complexity is increased since the components in these mixtures may act additively or synergistically. FIG. 2 provides a graph of data from a recent study illustrating a synergistic relationship between environmental tobacco smoke (ETS) and ambient levels of ozone (“O₃”). The graph illustrates the Penh rates, or airway resistance, following exposure of asthmatic rats to ETS, O₃, and a combination of both substances. Asthmatic rats were exposed to ETS for three hours at the rate of one cigarette every ten minutes, ambient equivalents levels of O₃ for three hours, or a combination of the two for three hours. FIG. 2 illustrates that exposure to ETS only for 3 hours did not significantly alter the Penh level in rats compared to the controls who were exposed to air (mean±SEM; 0.549±0.12 vs. 0.589±0.023; P=0.68). Ozone significantly increased Penh post exposure in rats compared to controls (0.773±0.063 vs. 0.589±0.023; P=0.04). Additionally, Ozone exposed rats also demonstrated significantly increased Penh values compared to ETS exposed rats (P=0.025). Furthermore, the combined exposure of ozone and ETS significantly increased Penh compared to either single exposure with ETS (0.971±0.081 vs. 0.549±0.012; P=0.0004) or ozone (0.773±0.063; P=0.033).

The ability of chemical substances to combine additively or synergistically, as shown in FIG. 2, makes diagnosis of triggers for asthma even more difficult, because the levels of chemical substances can vary dramatically over relatively small periods of time, even in the same environment. The data in Table 1 below illustrates the fluctuations between ozone concentration both inside and outside a residential home.

TABLE 1
Indoor/Outdoor Ozone Levels in a Residential Home

| Sample | Inside | Outside | | |
|---|---|---|---|---|
| Collection | Concentration | Concentration | Inside-Outside | % O3 |
| Time | (ppb) | (ppb) | (ppb) | Inside |
| 9:00 | 9 | 11 | 2 | 86 |
| 9:15 | 4 | 13 | 9 | 28 |
| 10:00 | 2 | 25 | 23 | 8 |
| 10:30 | 7 | 30 | 23 | 22 |
| 10:45 | 9 | 30 | 21 | 30 |
| 12:10 | 6 | 45 | 39 | 13 |
| 12:30 | 9 | 50 | 41 | 18 |
| 5:40 | 10 | 59 | 50 | 16 |
| 6:05 | 10 | 61 | 52 | 16 |
| 6:15 | 9 | 56 | 47 | 16 |
| 6:30 | 11 | 57 | 46 | 19 |

| | |
|---|---|
| Daily Avg % O3 Inside | 24 |

Conventional environmental monitoring systems fail to enable the level of analysis and data generation that is required to fully examine the complex range of variables that leads to many respiratory deficiencies, such as asthma. Therefore, it would be advantageous to provide a portable environmental monitoring system that would enable data to be collected regarding a user's exposure to airborne analytes in almost any environment.

Additionally, it would be advantageous to provide a portable environmental monitoring system configured not only to collect data regarding a user's exposure to airborne analytes but also capable of providing information regarding a user's pulmonary function.

Additionally, it would be advantageous to provide an improved system and method for diagnosing a respiratory deficiency trigger in which concomitant relationships can be established between exposure of a user to airborne analytes and a deficient pulmonary function.

### BRIEF SUMMARY OF THE INVENTION

The present invention describes systems and methods for providing portable environment monitoring systems. An exemplary embodiment of the present invention provides a portable environment monitoring system comprising a sensor enabled to sense an airborne analyte. The portable environment monitoring system also includes a microprocessor in communication with the sensor and enabled to process information received from the sensor. Additionally, the portable environment monitoring system includes a memory device in communication with the microprocessor and enabled to store information received from the microprocessor. Furthermore, a user is enabled to ambulate with the portable environment monitoring system.

In addition to portable environment monitoring systems, the present invention provides a method for diagnosing a respiratory deficiency trigger including providing a user with a portable environment monitoring system comprising a sensor, a microprocessor, a memory device, and a respiratory monitoring device. The method further includes collecting a plurality of data from the sensor and the respiratory monitoring device with the user in a plurality of environments and analyzing the plurality of data received from the sensor and the respiratory monitoring device. Additionally, the method for diagnosing a respiratory deficiency trigger includes determining whether a relationship exists between the exposure of the user to an airborne analyte and a deficient pulmonary function by the user.

These and other objects, features and advantages of the present invention will become more apparent upon reading the following specification in conjunction with the accompanying drawing figures.

### BRIEF DESCRIPTION OF THE FIGURES

FIG. 1 illustrates the percentages of asthma case rates in categories of age, race, and gender.

FIG. 2 provides a graph of data illustrating a synergistic relationship between environmental tobacco smoke (ETS) and ambient levels of ozone (“O₃”).

FIG. 3A provides a block diagram illustration of a portable environment monitoring system 300 in accordance with an exemplary embodiment of the present invention.

FIG. 3B provides a block diagram illustration of an alternative embodiment of the portable environment monitoring system 300 in accordance with the present invention.

FIG. 4 provides a block diagram illustration of a user provided with a portable environment monitoring system 300 in accordance with an exemplary embodiment of the present invention.

FIGS. 5A and 5B provide illustrations of a portable environment monitoring system 300 in accordance with an exemplary embodiment of the present invention.

FIG. 6 provides a schematic of a portable environment monitoring system 300 in accordance with an exemplary embodiment of the present invention.

FIG. 7 provides an illustration of a block diagram of the method for diagnosing a respiratory deficiency trigger 700 in accordance with an exemplary embodiment of the present invention.

FIG. 8 provides a graph of analysis of information obtained from an exemplary embodiment of the portable environment monitoring system 300 with an O₃ sensor 310E in accordance with an exemplary embodiment of the present invention.

FIG. 9 provides a graph of analysis of information obtained from an exemplary embodiment of the portable environment monitoring system 300 with a VOC sensor 310A and a CO₂ sensor 310B in accordance with an exemplary embodiment of the present invention.

FIG. 10 provides a graph of analysis of information obtained from an exemplary embodiment of the portable environment monitoring system 300 with a NO₂ sensor 310D in accordance with an exemplary embodiment of the present invention.

FIG. 11 provides a graph of analysis of this user field test of an exemplary embodiment of the portable environment monitoring system 300, including a VOC sensor 310A, a CO₂ sensor 310B, a formaldehyde sensor 310C, and a NO₂ sensor 310D.

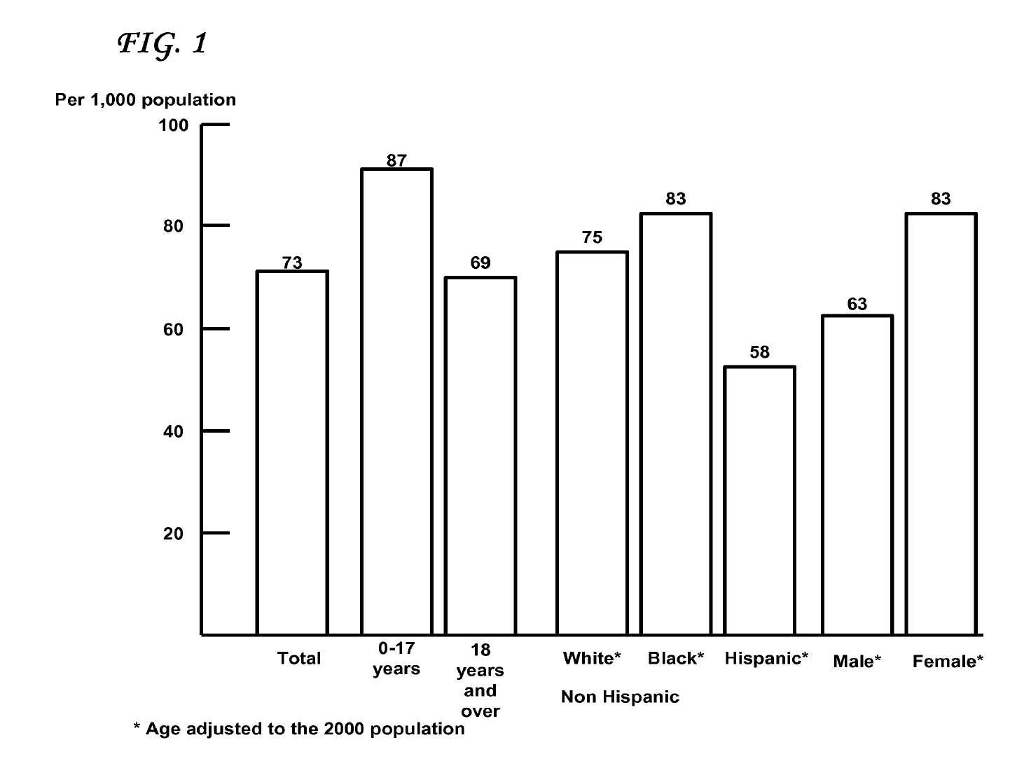

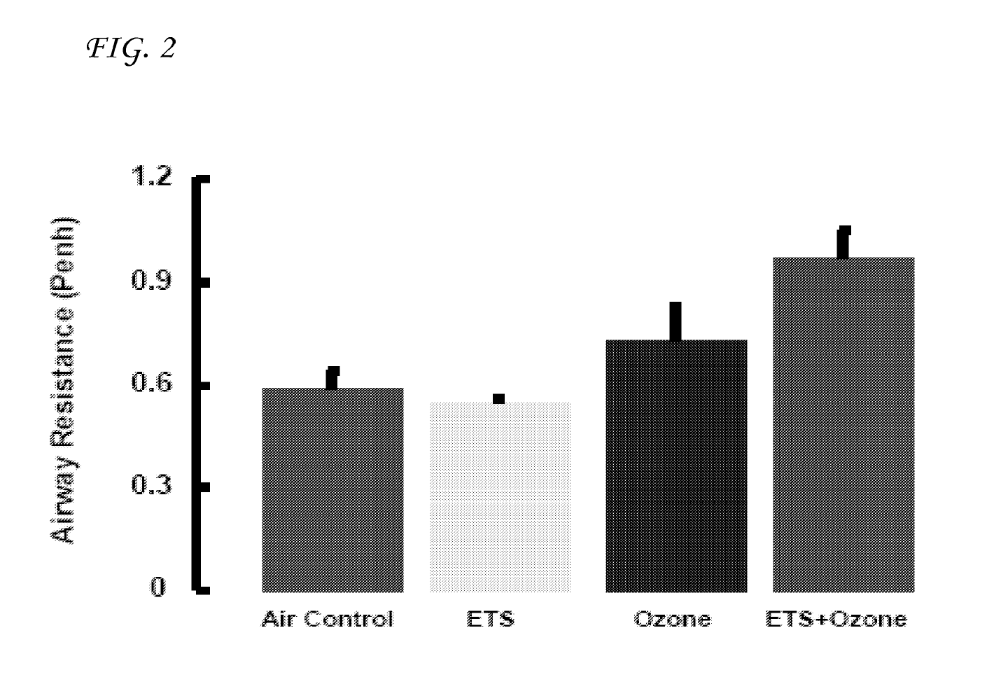

### DETAILED DESCRIPTION

The present invention addresses the deficiencies in the prior art concerning the inability to provide systems capable of monitoring airborne analytes. Significantly, the present invention provides methods and apparatus for providing portable environment monitoring systems. A portable environment monitoring system provided in accordance with the present invention is enabled to monitor the presence of one or more airborne analytes present in the environment of a user and store data regarding those substances. Additionally, the present invention overcomes the drawbacks of the conventional methods and systems in the prior art and provides systems and methods which can be conveniently carried and operated by the user in a variety of situations and environments.

An exemplary embodiment of the present invention provides a portable environment monitoring system comprising a sensor enabled to sense an airborne analyte. The portable environment monitoring system also includes a microprocessor in communication with the sensor and enabled to process information received from the sensor. Additionally, the portable environment monitoring system includes a memory device in communication with the microprocessor and enabled to store information received from the microprocessor. Furthermore, a user is enabled to ambulate with the portable environment monitoring system.

In addition to portable environment monitoring systems, the present invention provides a method for diagnosing a respiratory deficiency trigger including providing a user with a portable environment monitoring system comprising a sensor, a microprocessor, a memory device, and a respiratory monitoring device. The method further includes collecting a plurality of data from the sensor and the respiratory monitoring device with the user in a plurality of environments and analyzing the plurality of data received from the sensor and the respiratory monitoring device. Additionally, the method for diagnosing a respiratory deficiency trigger includes determining whether a relationship exists between the exposure of the user to an airborne analyte and a deficient pulmonary function by the user.

The portable environment monitoring systems enabled by the present invention present significant advantages to the area of asthmatic reaction analysis. Conventional monitoring instrumentation permits monitoring of very limited set of airborne analytes. Furthermore, conventional monitoring instrumentation is not portable. Typically, conventional monitoring systems require sophisticated and trained personnel to install and configure these permanently fixed machines. For example and not limitation, conventional monitoring systems require one or more sensory devices, a computer, a power source, a monitor, and additional equipment. Most often, these conventional monitoring systems require an independent and separate sensory device for measuring each individual airborne contaminant.

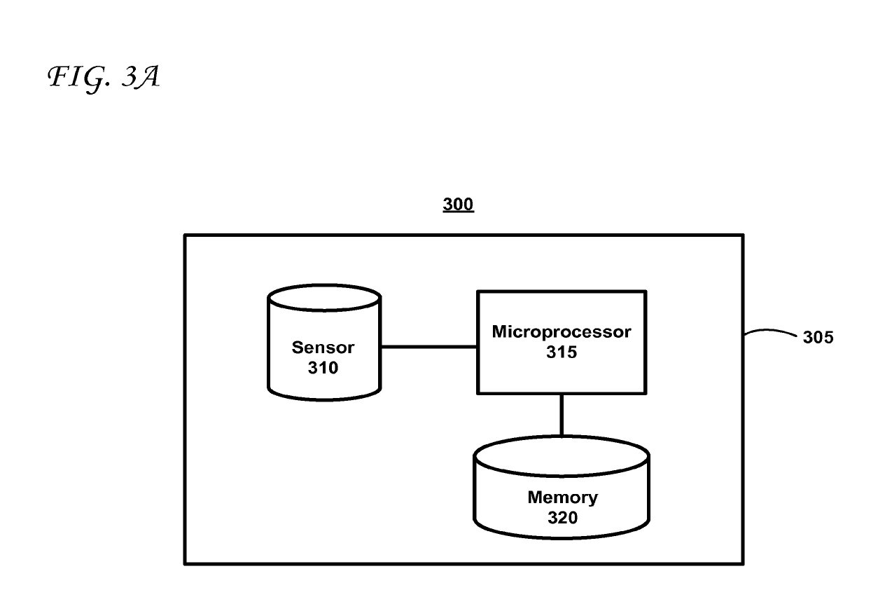

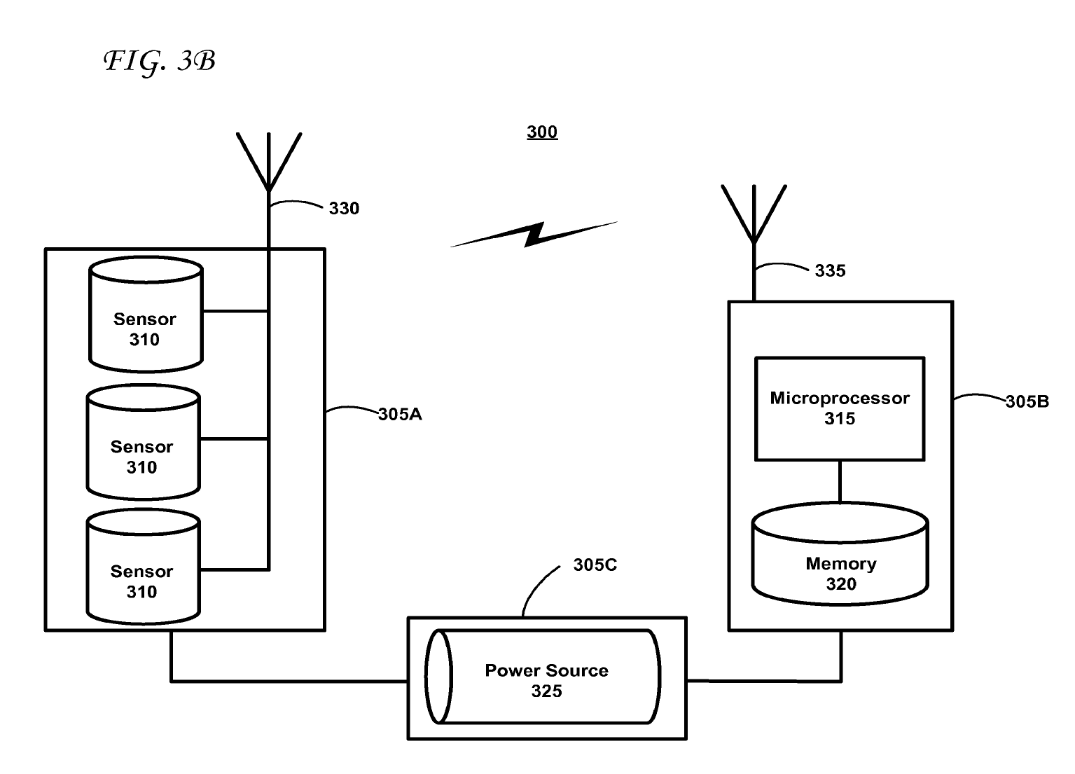

FIG. 3A provides a block diagram illustration of a portable environment monitoring system 300 in accordance with an exemplary embodiment of the present invention. As shown in the exemplary embodiment of FIG. 3A, the portable environment monitoring system 300 can provide a housing 305. In an exemplary embodiment, the housing 305 can provide the chassis for the components of the portable environment monitoring system 300. The housing 305 in an exemplary embodiment is comprised of a lightweight and sturdy material that is amenable to handling and portability by the user and also provide sufficient protection for the components of the portable environment monitoring system 300. Those of skill in the art will appreciate that the housing 305 can be made from a variety of suitable materials, including lightweight polymers and metals.

As shown FIG. 3A, an exemplary embodiment of the portable environment monitoring system 300 can provide a sensor 310. The sensor 310 can be enabled to sense one or more airborne analytes in an exemplary embodiment. The term analyte is used herein to describe any particle, chemical substance, compound, or other material. Therefore, the sensor 310 can be enabled to sense an airborne analytes proximate the portable environment monitoring system 300. Those of skill in the art will appreciate that the sensors described herein can be commercially available sensors or proprietary sensors developed specifically for the present invention. In an exemplary embodiment, the sensor 310 is configured in communication with a microprocessor 315. The microprocessor 315 depicted in FIG. 3A can be enabled to receive data or information generated by the sensor 310. The features of the microprocessor 315 can vary upon the demands of a particular implementation of the portable environment monitoring system 300. Those of skill in the art will appreciate that the microprocessor 315 could be Application Specific Integrated Circuit (“ASIC”) specifically designed for one embodiment of the portable environment monitoring system 300 or a generic microprocessor configured for operation in a variety of different embodiments of the portable environment monitoring system 300. An exemplary embodiment of the portable environment monitoring system 300 implements a microprocessor 315 with a relatively compact and small footprint and minimal power consumption so as to aid in minimizing the power and space requirements of the portable environment monitoring system 300.

As shown in the block diagram in FIG. 3A, an exemplary embodiment of the portable environment monitoring system 300 further includes a memory device 320. The memory device 320 is provided in communication with the microprocessor 315. In an exemplary embodiment of the portable environment monitoring system 300, the memory device 320 is enabled to store data processed by the microprocessor 315 and received from the sensor 310. In an exemplary embodiment, the memory device 320 is a low power, compact component that provides non-volatile storage. Therefore, in the exemplary embodiment, the memory device 320 may retain stored data even if the power to the portable environment monitoring system 300 is lost. Those of skill in the art will appreciate that a variety of memory devices can be implemented for the memory device 320 to satisfy the requirements of particular embodiments of the portable environment monitoring system 300. In an exemplary embodiment, the portable environment monitoring system 300 is configured such that the sensor 310 can gather information regarding the presence of one or more airborne analytes in the environment of the portable environment monitoring system 300 and communicate that information regarding the one or more airborne analytes to the microprocessor 315. The microprocessor 315, in an exemplary embodiment of the portable environment monitoring system 300, is enabled to control the sensor 310. For example and not limitation, the microprocessor 315 can control, when the sensor 310 is engaged, how long the sensor 310 is operable, and how much information is gathered by the sensor 310. Furthermore, the microprocessor 315 is enabled to communicate with the memory device 320 such that data generated by the sensor 310 can be processed by the microprocessor 315 and stored on the memory device 320.

An exemplary embodiment of the portable environment monitoring system 300 provides a relatively lightweight and compact system and enables the user to ambulate with the system 300. In some embodiments the specific total weight of the portable environment monitoring system 300 is less than five pounds and preferably less than one pound. Therefore, the user is enabled to carry and/or wear the portable environment monitoring system 300 in almost any environment and while engaging a wide variety of tasks. For example, and not limitation, the user can wear the portable environment monitoring system 300 around the home, school, and/or office. Furthermore, the user can wear the portable environment monitoring system 300 outside and when walking, going up steps, and engaging in certain physical activities. The portability and convenience of portable environment monitoring system 300 enables many of significant advantages of the present invention with regard to the ability of system to monitor the user in a wide variety of different environments at all times of the day.

FIG. 3B provides a block diagram illustration of an alternative embodiment of the portable environment monitoring system 300 in accordance with the present invention. The embodiment of the portable environment monitoring system 300 shown in FIG. 3A illustrates an implementation in which the components of the portable environment monitoring system 300 are contained in one housing 305. As shown in the alternative embodiment illustrated in FIG. 3B, the portable environment monitoring system 300 can also be separated into multiple component housings, including 305A, 305B, and 305C. As shown in the alternative embodiment in FIG. 3B, the sensors 310 can be configured in a separate housing 305A. Furthermore, the sensor housing 305A can be equipped in this alternative embodiment with an antenna 330 to enable wireless transmission of the sensory information to the microprocessor 315 and the memory device 320 and enable the reception of instructions from the microprocessor 315. Similarly, as shown in the alternative embodiment in FIG. 3B, the portable environment monitoring system 300 can be configured with the microprocessor 315 and the memory device 320 in a separate housing 305B. This housing 305B can also be configured with an antenna 335 to wirelessly transmit and receive data from a variety of sources, including the sensors 310 and external computing equipment. For example, and not limitation, the microprocessor 315 of this alternative embodiment of the portable environment monitoring system 300 can be configured to receive commands and controls from a remote wireless source and can be configured to receive new firmware uploads and updates from a remote wireless source, such as a laptop. In addition to separating the processing components in the alternative embodiment of the portable environment monitoring system 300 shown in FIG. 3B, the power source 325 can also be separated into its own housing 305C. For example, and not limitation, the power source 325 in housing 305C is often a heavier component of the portable environment monitoring system 300 and could be configured to be tucked away in a backpack, fanny pack, or pocket of the user. Although, the portable environment monitoring system 300 shown in FIG. 3B is configured with antennas 330 and 335 for wireless operation, it can also be configured for wired communication. Those of skill in the art will appreciate that there a variety of suitable ways to divide and configure the various embodiments of the portable environment monitoring system 300, depending on the demands and requirements of a given implementation. Those of skill in the art will further appreciate that the portable environment monitoring system 300 can be configured in these different ways without detracting from the scope of the invention.

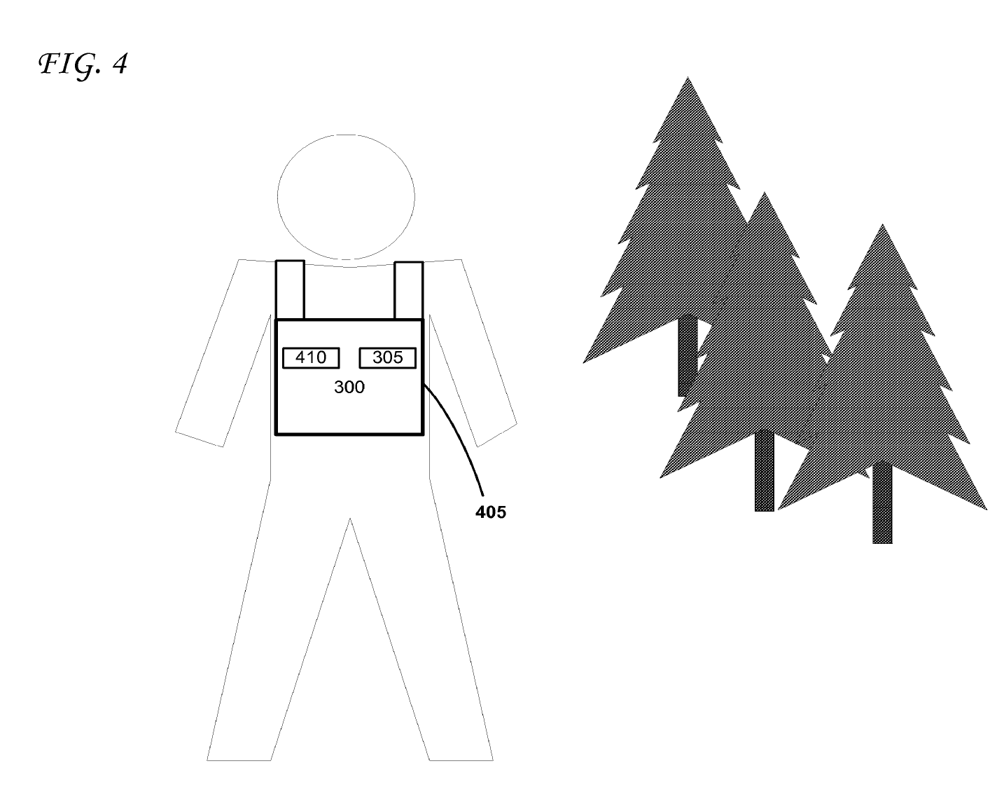

FIG. 4 provides a block diagram illustration of a user provided with a portable environment monitoring system 300 in accordance with an exemplary embodiment of the present invention. The exemplary embodiment of the portable environment monitoring system 300 depicted in FIG. 4 is configured to be conveniently carried by a user. Therefore, the portable environment monitoring system 300 is sufficiently lightweight such that it can be carried in a piece of clothing, pack, or pocket of the user. An exemplary embodiment of the portable environment monitoring system 300 is less than five pounds, and preferably less than two pounds. In the exemplary embodiment depicted in FIG. 4, the portable environment monitoring system 300 is configured to be worn in a garment 410. The garment 410 can be a variety of different types of clothing, including a vest, a jacket, pants or a shirt. The garment 410 can be designed to be an undergarment or outerwear.

In alternative embodiment, the portable environment monitoring system 300 can be configured to fit into a backpack or fanny pack to be worn by the user. Furthermore, some embodiments of the portable environment monitoring system 300 are configured with separate and discrete components such that the system 300 can be broken into discrete components to be worn or concealed in various pockets and packs. For example, the power source of the portable environment monitoring system 300 could be stored in a fanny pack, while the microprocessor 315, sensor 310, and memory device 320, are stored in a pocket of a user's vest garment 410. As depicted in FIG. 4, the user can be enabled to wear the portable environment monitoring system 300 in a variety of environments, including outdoors.

One of the significant advantages of an exemplary embodiment of the present invention is that it enables a user to monitor airborne analytes in a variety of environments. Conventional environmental monitoring systems are fixed and bulky apparatus that require a relatively significant amount of space and a relatively large power source. Thus, conventional environmental monitoring equipment can only monitor the room in which they are located. Typically, the conventional environmental monitoring equipment is setup in a user's hospital room or bedroom. In this manner, data can only be analyzed with respect to the user's exposure to airborne analytes in proximity to the conventional fixed environmental monitoring device. If analysis is desired of a different environment, the conventional system must be disassembled and reconfigured in another room. Additionally, the user is not enabled by the conventional systems to gather data regarding exposure to airborne analytes in outdoor environments. As depicted in FIG. 4, the user can wear the portable environment monitoring system 300 in almost any environment, including outdoors. Therefore, the portable environment monitoring system 300 enables a user to gather data concerning airborne exposures in a large range of environments over significant periods of time.

An additional, significant advantage of an exemplary embodiment of the portable environment monitoring system 300 is that it enables real-time collection of airborne analyte exposure data. As shown in Table 1 above, the level of particles entrained in the air in a given outdoor or indoor environment can vary greatly even in a 24-hour period. An exemplary embodiment of the portable environment monitoring system 300 enable the user not only to gather data in multiple environments but also gather data over extended and varying periods of time in those environments. Furthermore, airborne analytes may react with each other to modify the substances and/or create new airborne substances. For example, and not limitation, ambient O₃ may react with unsaturated compounds, such as those found in commonly used indoor cleaning products, and produce oxidated compounds. Therefore, the ability to obtain real time data is critical to determining relationships between certain airborne analytes and deficient user pulmonary function.

The exemplary embodiment of the portable environment monitoring system 300 shown in FIG. 4 includes a respiratory monitoring device 410. The respiratory monitoring device 410 can enable the user to collect data concerning the user's pulmonary function. The user's pulmonary function is an indication as to the efficacy of the user's lung function. The respiratory monitoring device 410 can be a variety of different types of devices enabled to perform Pulmonary Function Tests (“PFTs”). An exemplary embodiment of the respiratory monitoring device 410 is a peak flow meter. A peak flow meter is a small, hand-held device used to monitor a user's ability to breathe out air. A peak flow meter can measure the airflow through the bronchi and thus the degree of obstruction in the airways. In one embodiment, the respiratory monitoring device 410 is an PiKo-1 handheld device, manufactured by nSpire Health, Inc., which measures peak flow and Forced Expiratory Volume in 1 second (“FEV₁”). Those of skill in the art will appreciate that other spirometry devices can be implemented in the portable environment monitoring system 300 to provide data regarding the user's pulmonary function, including data regarding a user's Forced Viral Capacity (“FVC”), FEV₁, and Peak Expiratory Flow (“PEF”) data.

One of the significant advantages provided by an exemplary embodiment of the portable environment monitoring system 300 is that it enables analysis of both data concerning exposure of a user to an airborne analyte, but also determinative comparisons of airborne analyte exposure data with data regarding the pulmonary function of the user. Therefore, a portable environment monitoring system 300 can enable the determination of concomitant relationship between exposure of a user to a particular airborne analyte and a decrease in the user's pulmonary function.

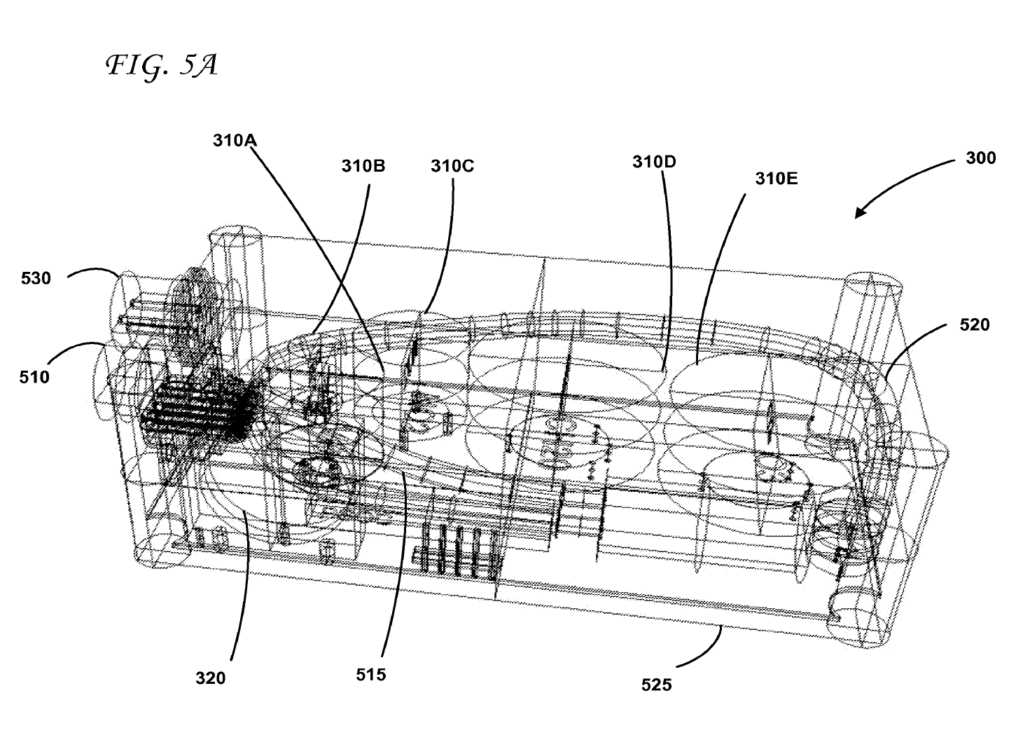

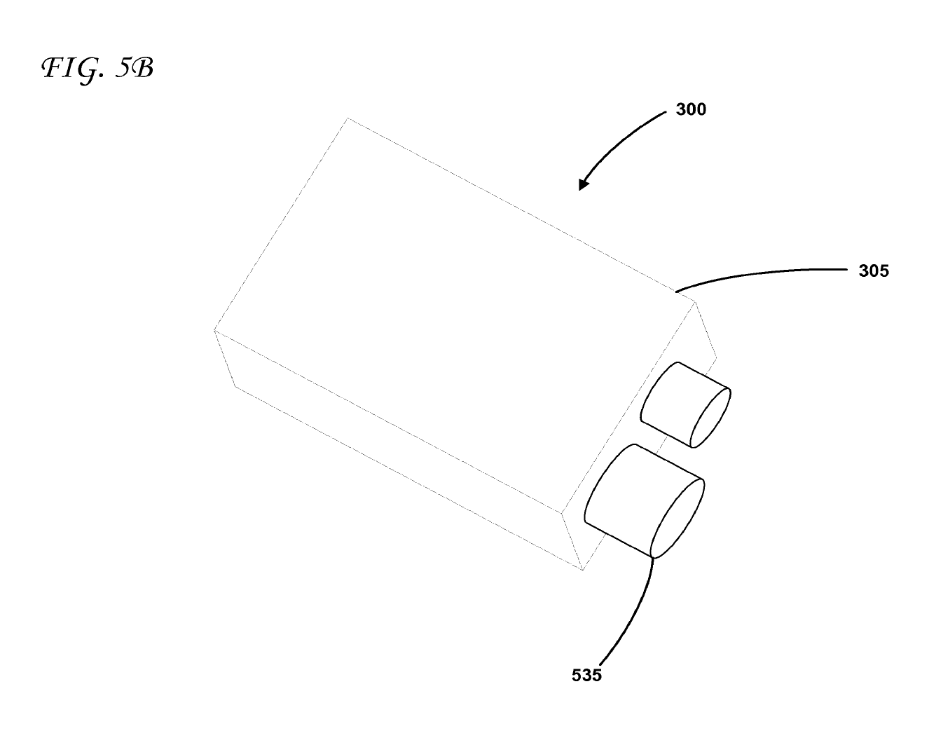

FIGS. 5A and 5B provide illustrations of a portable environment monitoring system 300 in accordance with an exemplary embodiment of the present invention. As shown in FIG. 5A, the portable environment monitoring system 300 can include a housing 305. This housing 305, in an exemplary embodiment, can be a rectangular volume. In the exemplary embodiment depicted in FIG. 5A, the housing 305 has a length of 4.75 inches, a width of 2.6 inches, and a height of 1.6 inches. Those of skill in the art will appreciate that the exemplary embodiment shown in FIG. 5A is just one implementation and that the dimensions of the housing 305 can vary according to the parameters of an embodiment of the portable environment monitoring system 300. The housing 305 of an exemplary embodiment can provide both a chassis for some of the components of the portable environment monitoring system 300 and one or more interfaces to external components.

In an exemplary embodiment of the portable environment monitoring system 300, an air pump 505 is configured within the housing 305. The air pump is enabled to draw ambient air through the air inlet 510 and the air inlet tube 515. In an exemplary embodiment, the air inlet 510 can provide a particulate filter 550 for filtering the ambient air. The particulate filter 550 in the air inlet 510 can be configured to permit only respirable size particles to be passed into the system. Therefore, the portable environment monitoring system 300 can be enabled to analyze only those airborne analytes that are of respirable size. The pump 505 can be configured to draw in ambient air and then pass that air over one or more sensors in the portable environment monitoring system 300. As shown in FIG. 5A, the pump can be connected to an air delivery tube 520. The air delivery tube 520 can be configured within the housing to direct the incoming and filtered ambient air over the sensor for detection.

In some embodiments of the portable environment monitoring system 300, the particulate filter 550 can be used to trap and contain particles below a certain size. For example, and not limitation, a particulate filter 550 can implemented to trap respirable-sized particulate matter of less than 2.5 micrometers in diameter and smaller (“PM_{2.5}”). In addition to the airborne analytes analyzed by the sensors 310, these exemplary embodiment of the portable environment monitoring system 300 also enable subsequent analysis of the particles trapped by the particulate filter 550. Therefore, the particulate filter 550 can be removed from the air inlet 510 after a series of tests with an exemplary embodiment of the portable environment monitoring system 300 and then laboratory analyzed for composition, including allergens and microbes.

As shown in FIG. 5A, an exemplary embodiment of the portable environment monitoring system 300 can include multiple sensors. The exemplary embodiment shown FIG. 5A provides five sensor devices for detecting a plurality of airborne analytes. Sensor 310A show in the exemplary embodiment in FIG. 5A is a Volatile Organic Compound (“VOC”) sensor. This VOC sensor 310A can be configured to measure a variety of different volatile organic compounds. Table 2 below provides a list of the some of the various VOCs which can be detected and/or measured by the VOC sensor 310A:

TABLE 2
Volatile Organic Compounds
(exemplary list)

Acetaldehyde
Acetone
Aliphatic Compounds (C₈-C₁₁)
Benzaldehyde
Benzene
1,3-Butadiene
Butanols (particularly 1-butanol)
Ethylbenzene
2-Ethyl-1-hexanol
Formaldehyde
PAHs (petroleum-based VOCs frequently from
traffic exposures)
Styrene
Terpenes (such as limoene and pinenes)
Tetrachloroethylene (carbon tetrachloride)
TXIB (2,2,4-trimethyl-1,3-pentadio diisobutyrate)
Texanol (2,2,4-trimethyl-1,3-pentanediol
monobutyrate)
Toluene
Xylenes

As shown in FIG. 5A, the air delivery tube 520 can be configured to deliver air over the intake area of VOC sensor 310A. In an exemplary embodiment, the VOC sensor 310A can relay information to the microprocessor 315 (not visible in FIG. 5A). The microprocessor 315 can be configured to process this information from the VOC sensor 310A. Furthermore, the microprocessor 315 can control the storage of data relating to the information received from the VOC sensor 310A in the memory device 320. As shown in FIG. 5A, the memory device 320 can be configured within the housing and proximate the sensors and microprocessor 315.

In addition to the VOC sensor 310A, the exemplary embodiment of the portable environment monitoring system 300 show in FIG. 5A provides a carbon dioxide (“CO₂”) sensor 310B. The CO₂ sensor 310B can be enabled to receive ambient air from the pump 505 and detect the presence of certain levels of carbon dioxide in the ambient air. Furthermore, the CO₂ sensor 310B can be configured in an exemplary embodiment of the portable environment monitoring system 300 in communication with the microprocessor 315 such that information is received from the CO₂ sensor 310B by the microprocessor 315. The exemplary embodiment of the portable environment monitoring system 300 in FIG. 5A also provides a formaldehyde sensor 310C. Similar to other sensors, formaldehyde sensor 310C can be configured in communication with the microprocessor 315 to provide information regarding the detection of certain levels of formaldehyde in the environment of an exemplary embodiment of the portable environment monitoring system 300.

The exemplary embodiment of the portable environment monitoring system 300 shown in FIG. 5A further includes a nitrogen dioxide (“NO₂”) sensor 310D and an ozone or trioxygen (“O₃”) sensor 310E. Similar to the other sensors, both the NO₂ sensor 310D and the O₃ sensor 310E are configured in communication with the microprocessor 315 to provide information regarding the detection of certain levels of nitrogen dioxide and ozone in the environment of an exemplary embodiment of the portable environment monitoring system 300. Those of skill in the art will appreciate that additional sensors can be added to portable environment monitoring system 300 without detracting from the scope of the invention. Furthermore, sensors can be omitted from the portable environment monitoring system 300 in accordance with the type of analytes that are to be monitored by the system.

In an exemplary embodiment, the portable environment monitoring system 300 is battery powered. In the exemplary embodiment shown in FIG. 5B, batteries are configured on the circuit board 525 and connected to a power connector 530. Therefore, in an exemplary embodiment, the batteries of the portable environment monitoring system 300 can be recharged by connecting a power source to the power connector 530. As shown in FIG. 5B, the portable environment monitoring system 300 can further provide a data interface connector 535. The data interface connector 535 can enable an exemplary embodiment of the portable environment monitoring system 300 to transmit and receive data from an external device. For example, and not limitation, in the exemplary embodiment shown in FIG. 5B, the data interface connector 535 is a serial port that be connected to an external computer. In this embodiment, once a serial cable is connected to the data interface connector 535, data stored in the memory device 320 of the portable environment monitoring system 300 can be downloaded to an external device. The data output by the portable environment monitoring system 300 can be in a variety of forms, including a Microsoft Excel file or other database file, enabling convenient and expedient processing and analysis of the data.

One significant advantage of an exemplary embodiment of the portable environment monitoring system 300 is that it can be enabled to output data directly from the portable system 300. Therefore, unlike conventional systems that typically require the sensor components to be individually connected to an external computer, an exemplary embodiment of the portable environment monitoring system 300 can process the sensor information and generate a data file for output. The microprocessor 315 of the portable environment monitoring system 300 can be configured, in an exemplary embodiment, to receive and process information from the sensors and store it on the memory device 320 in a desired data output format, such as a Microsoft Excel data file.

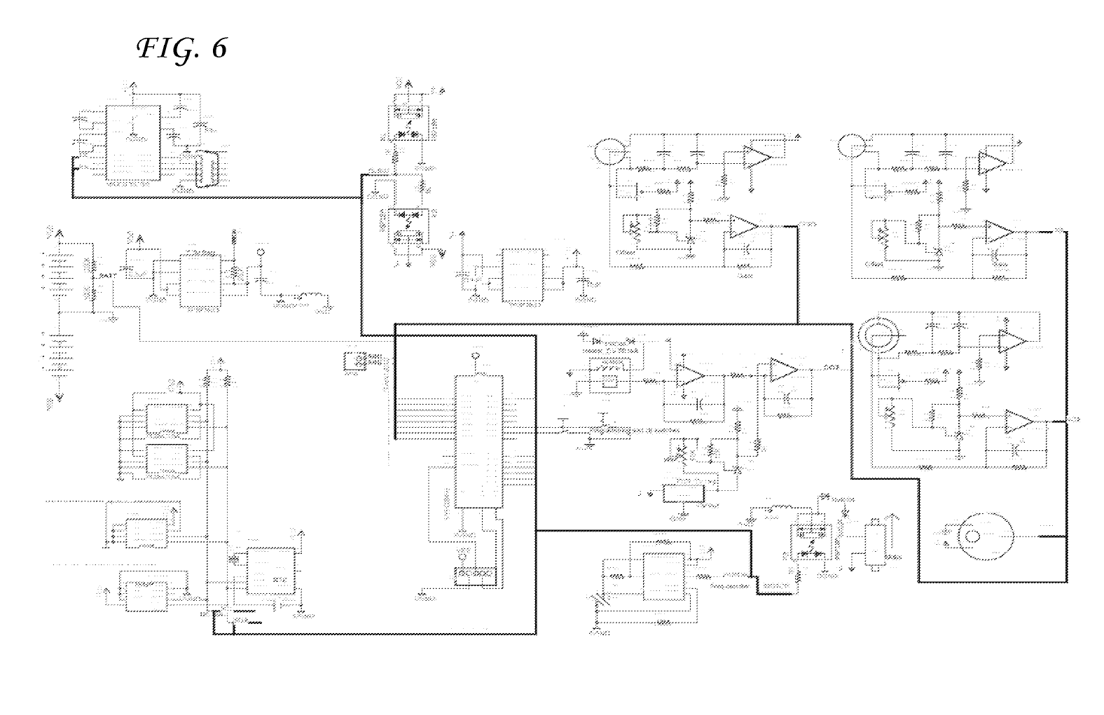

FIG. 6 provides a schematic of a portable environment monitoring system 300 in accordance with an exemplary embodiment of the present invention. The schematic of the exemplary embodiment of the portable environment monitoring system 300 shown in FIG. 6 provides the layout and interconnections between the microprocessor 315, the memory device 320, and the sensors 310.

The microprocessor 315 can execute a particular load of firmware according to a particular embodiment of portable environment monitoring system 300, providing the necessary functions and instructions for the microprocessor 315 to control the operation of the portable environment monitoring system 300. In an exemplary embodiment, the microprocessor 315 is enabled to perform a variety of functions. For example, and not limitation, the microprocessor 315 can be configured to receive analog signals from the sensors 310 and perform an analog to digital conversion of those signals. In some embodiments, the microprocessor 315 relies upon an analog-to-digital conversion device to perform the signal conversion. Once the analog signals have been converted to a digital representation, the microprocessor 315 in an exemplary embodiment can be enabled to process those digital signals. For example, and not limitation, in a power-saving operation mode, the ambient air in the environment is monitored at regular intervals; thus, the microprocessor 315 can be configured to power-up and power-down the circuitry when needed. In an exemplary embodiment, the microprocessor 315 can be configured to control the operation of the sensors 310. Furthermore, the microprocessor 315 in an exemplary embodiment can control the pump 505 to determine when the pump draws in ambient air and how long the pump 505 operates. Additionally, the microprocessor 315 in an exemplary embodiment can control the transmission and reception of data via the data interface connector 535.

In an exemplary embodiment, the microprocessor 315 can execute firmware that enables the portable environment monitoring system 300 to operate in number of different modes. For example, and not limitation, the microprocessor 315 can require portable environment monitoring system 300 to operate in a “power-saving” mode in one setting and, in another setting, the microprocessor 315 can require the portable environment monitoring system 300 to be in a “always-on” mode where monitoring is continuous. Additionally, the microprocessor 315 in an exemplary embodiment might be equipped to operate in a “user command” mode, such that the portable environment monitoring system 300 is in operation only when the user has the system 300 powered-up. Those of skill in the art will appreciate that the firmware for the microprocessor 315 can vary from implementation to implementation and can provide a wide variety of operation modes and feature sets for embodiments of the portable environment monitoring system 300.

In the exemplary embodiment in which the portable environment monitoring system 300 operates in a “power-saving” mode, the system 300 is configured to automatically initialize the sensor on power-up and then transmit any previously recorded data to the data interface connector 535 for which transmission is desired. Furthermore, the system 300 is configured to power-up and conduct monitoring at regular intervals, such as two minute, three minute, or twenty minute intervals. Upon waking from a sleep mode, the exemplary embodiment of the portable environment monitoring system 300 powers the sensors 310, and then begins to power the air pump 505 to drawn in ambient air from the environment. The sensors 310 can then perform a test of the ambient air and output signals to be processed and stored in the memory device 320.

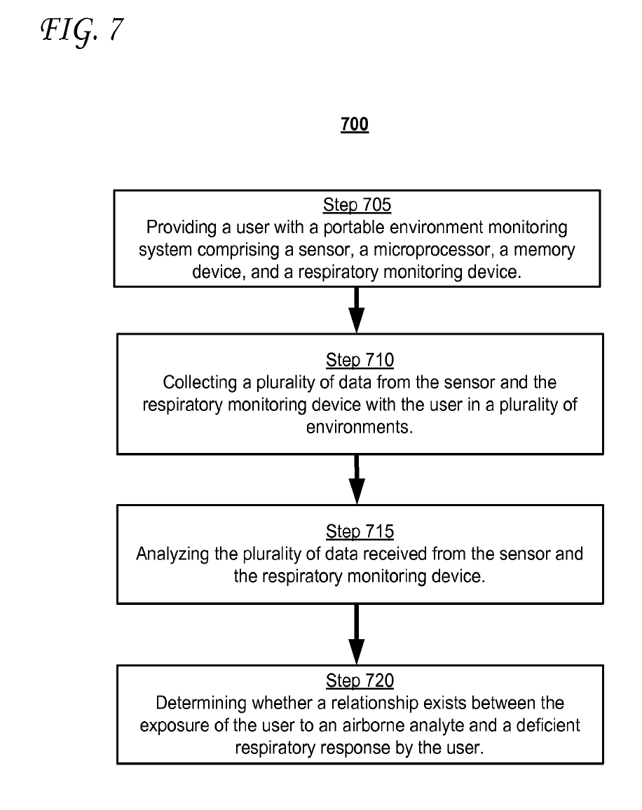

FIG. 7 provides an illustration of a block diagram of the method for diagnosing a respiratory deficiency trigger 700 in accordance with an exemplary embodiment of the present invention. The term respiratory deficiency trigger is used herein to describe an airborne analyte, which results in a decease in pulmonary function upon exposure of the user to the airborne analyte. As shown in FIG. 7, the first step 705 of an exemplary embodiment of the method for diagnosing a respiratory deficiency trigger 700 involves providing a user with a portable environment monitoring system comprising a sensor, a microprocessor, a memory device, and a respiratory monitoring device. The second step 710 of an exemplary embodiment of the method for diagnosing a respiratory deficiency trigger 700 involves collecting a plurality of data from the sensor and the respiratory monitoring device with the user in a plurality of environments. The third step 720 of an exemplary embodiment of the method for diagnosing a respiratory deficiency trigger 700 involves analyzing the plurality of data received from the sensor and the respiratory monitoring device. The fourth step 725 of an exemplary embodiment of the method for diagnosing a respiratory deficiency trigger 700 involves determining whether a relationship exists between the exposure of the user to an airborne analyte and a deficient pulmonary function by the user.

In an exemplary embodiment of the method for diagnosing a respiratory deficiency trigger 700, the deficient pulmonary function by the user corresponds to the user experiencing an asthma attack. Thus, the method for diagnosing a respiratory deficiency trigger 700 involves obtaining data regarding both the deficient pulmonary function by the user (i.e., the onset of the asthma attack) and obtaining data regarding the airborne analytes to which the user was exposed around the time of the asthma attack. Through analysis of the data obtained, relationships can be drawn between a user's exposure to a particular airborne analyte and the onset of an asthma attack. For example, and not limitation, analysis of data can result in a determination of a concomitant relationship between high levels of ozone exposure and user's asthma attack, in the event that the data obtained from the respiratory monitoring device and an ozone sensor indicates that the timing of the asthma attack corresponds to a certain level of ozone exposure. Those of skill in the art will appreciate that an asthma attack is just one form of a deficient pulmonary function and the method for diagnosing a respiratory deficiency trigger 700 can be used to detect a large variety of respiratory deficiencies, including Chronic Obstruction Pulmonary Disease (“COPD”), chronic bronchitis, pulmonary fibrosis, and sarcoidosis.

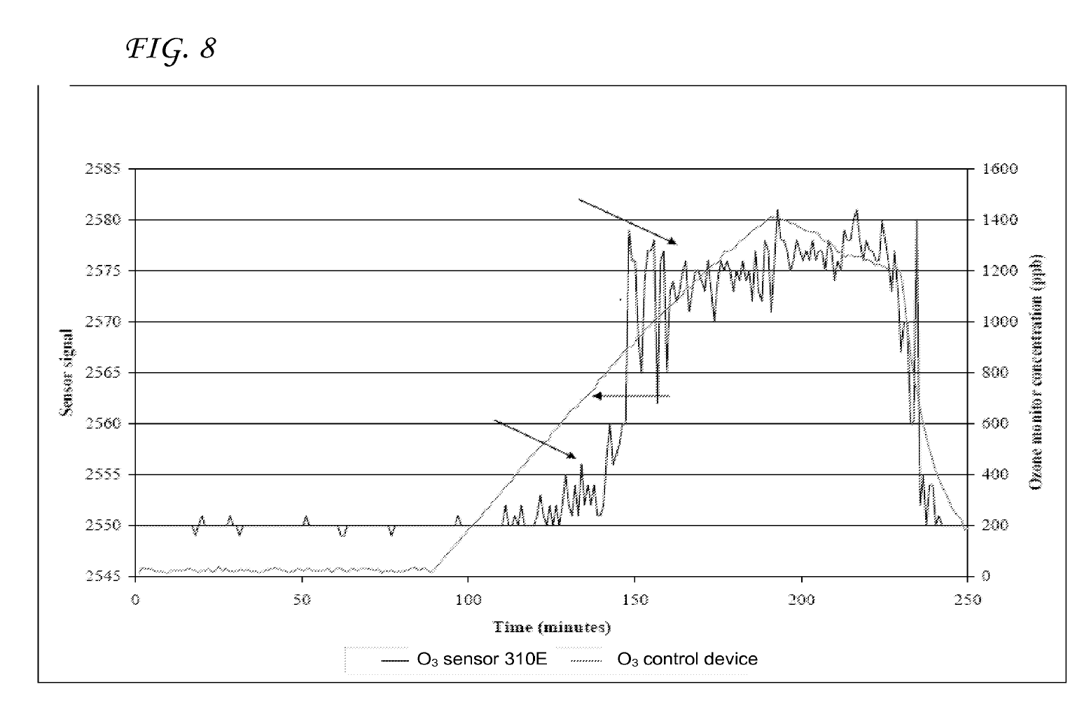

FIG. 8 provides a graph of analysis of information obtained from an exemplary embodiment of the portable environment monitoring system 300 having an O₃ sensor 310E in accordance with an exemplary embodiment of the present invention. The graph shown in FIG. 8 provides a graph of the output of the levels of ozone detected by the O₃ sensor 310E of the portable environment monitoring system 300 in accordance with an exemplary embodiment of the present invention. The jagged line shown in FIG. 8 provides the output signal of the exemplary embodiment of the O₃ sensor 310E in correspondence with the smooth line response of an ozone monitor device provided as a calibration control for the particular embodiment of the portable environment monitoring system 300 shown in FIG. 8. As shown in FIG. 8, the O₃ sensor 310E in this particular embodiment of the portable environment monitoring system 300 is capable of measuring levels of ozone above around 725 parts per billion (“ppb”). The Level of Detection (“LOD”) of a particular sensor 310 in a particular embodiment of the portable environment monitoring system 300 will vary upon the capability of the sensor 310. Those of skill in the art will appreciate that a variety of different sensors 310 with different LOD capabilities can be implemented in various embodiments of the portable environment monitoring system 300 without detracting from the scope of the invention.

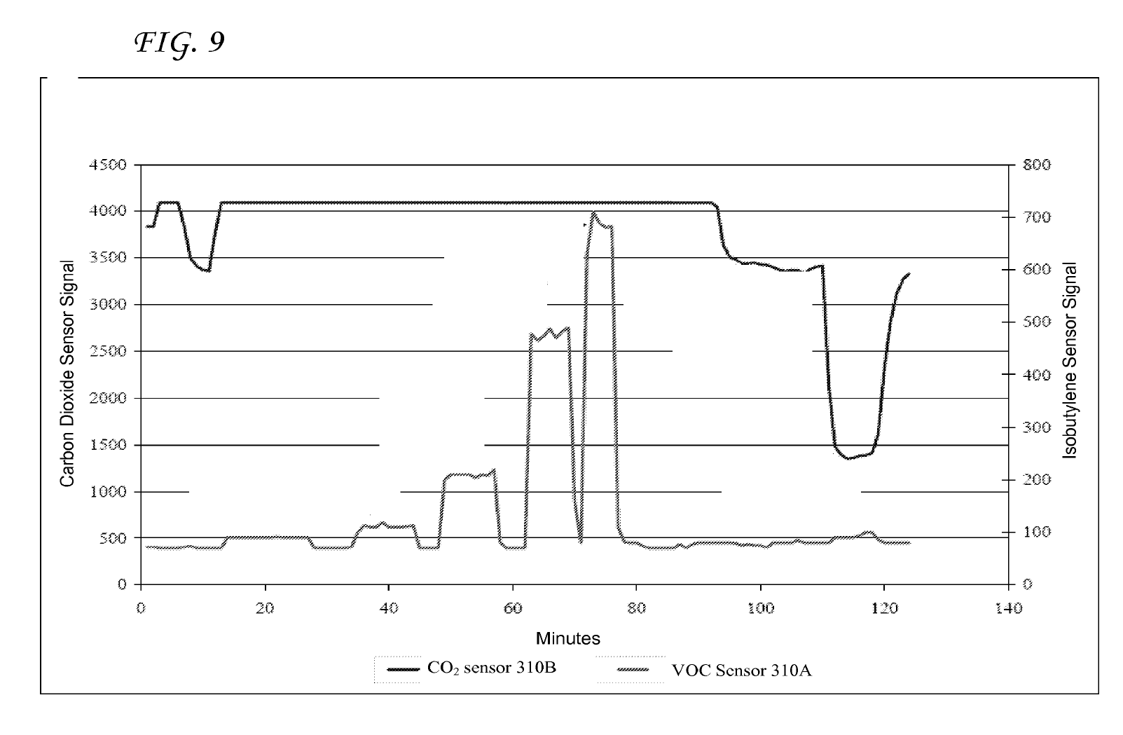

FIG. 9 provides a graph of analysis of information obtained from an exemplary embodiment of the portable environment monitoring system 300 with a VOC sensor 310A and a CO₂ sensor 310B in accordance with an exemplary embodiment of the present invention. The graph shown in FIG. 9 provides a graph of the output of the levels of carbon dioxide detected by the CO₂ sensor 310B of the portable environment monitoring system 300 in accordance with an exemplary embodiment of the present invention. As shown in FIG. 9, the CO₂ sensor 310B in this particular embodiment of the portable environment monitoring system 300 is capable of measuring levels of ozone above around 350 parts per million (“ppm”). Therefore, this embodiment of the portable environment monitoring system 300 can deliver viable information regarding CO2 levels above 350 ppm in the environment of the user. Furthermore, as shown in FIG. 9, the VOC sensor 310A in this particular embodiment of the portable environment monitoring system 300 is capable of detecting certain VOCs, isobutylene in this particular example, at levels above around 10 ppb.

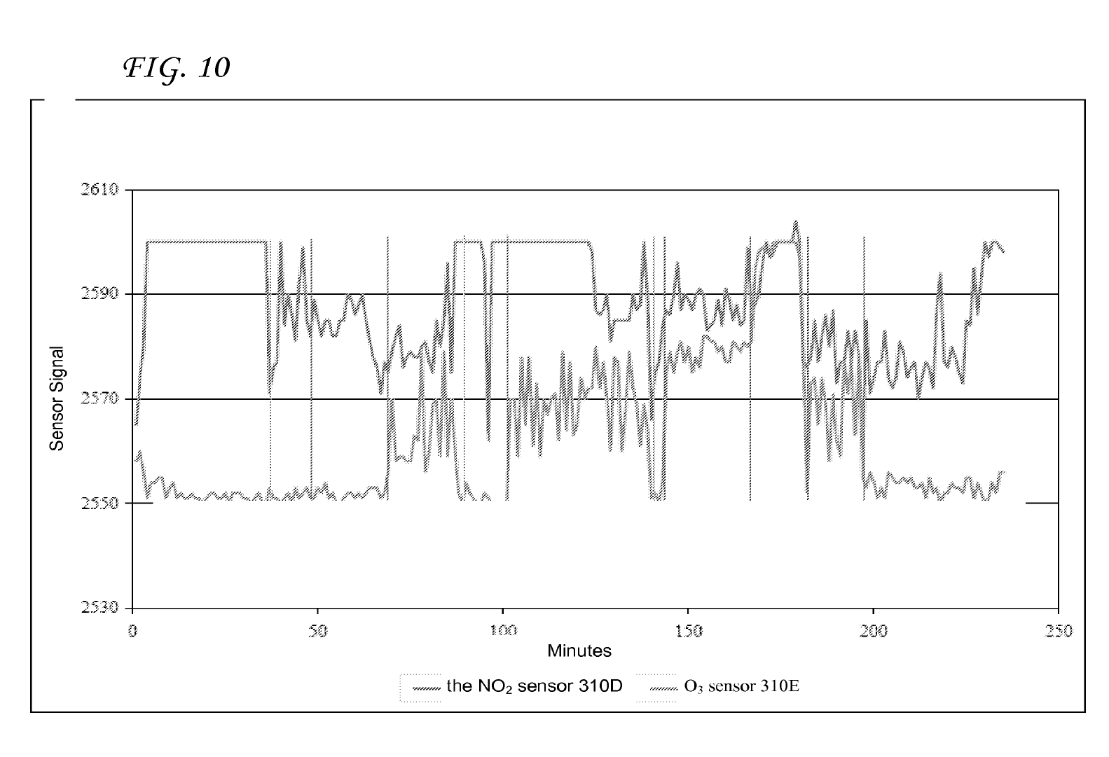

FIG. 10 provides a graph of analysis of information obtained from an exemplary embodiment of the portable environment monitoring system 300 with a NO₂ sensor 310D in accordance with an exemplary embodiment of the present invention. The graph shown in FIG. 10 provides a graph of the output of the levels of nitrogen dioxide detected by the NO₂ sensor 310D of the portable environment monitoring system 300 in accordance with an exemplary embodiment of the present invention. As shown in FIG. 10, the NO₂ sensor 310D in this particular embodiment of the portable environment monitoring system 300 is capable of measuring levels of ozone above around 140 ppb. Therefore, this embodiment of the portable environment monitoring system 300 can deliver viable information regarding NO₂ levels above 140 ppm in the environment of the user.

One of the significant advantages of the present invention is that it enables a user to track and obtain information regarding the airborne analytes to which the user is exposed in a large variety of environments. For example, and not limitation, an exemplary embodiment of portable environment monitoring system 300 was used in a particular field test to examine and analyze the users' exposures to a variety of airborne analytes monitored by the sensors 310 of the system 300. The data generated by the field test of this exemplary embodiment of the portable environment monitoring system 300 was then outputted to an external computer and analyzed.

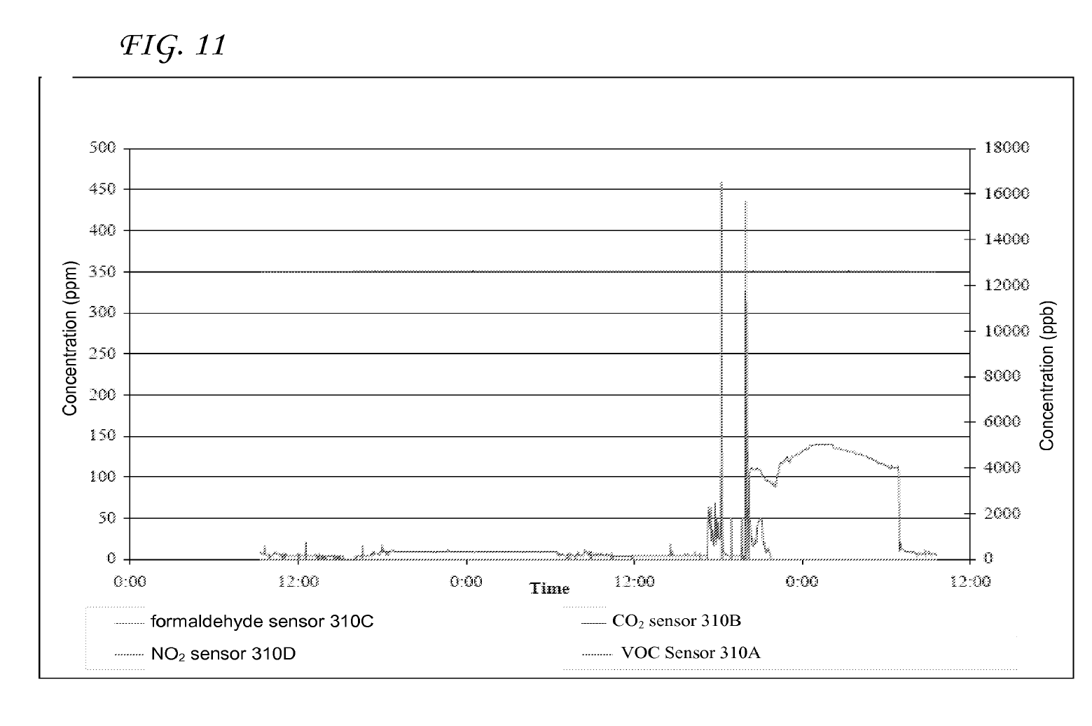

FIG. 11 provides a graph of analysis of this user field test of an exemplary embodiment of the portable environment monitoring system 300, configured with a VOC sensor 310A, a CO₂ sensor 310B, a formaldehyde sensor 310C, and a NO₂ sensor 310D. The graph shown in FIG. 11 provides data generated from each of the sensors 310. The only airborne analyte shown to have a notable response in the graph of FIG. 11 is a relatively high level of detection by the VOC sensor 310A of the exemplary embodiment of the portable environment monitoring system 300. The sensory data graphed in FIG. 11 is shown over the course of a twenty-four hour period, corresponding to the user's day. The graph in FIG. 11 illustrates that at some point in the evening, around 5:00 to 6:00 PM, the user was exposed to a relatively high level of VOC. Furthermore, the VOC exposure would remain elevated, as shown by the rectangular shaped chart line on the right hand side of the graph in FIG. 11, until sometime in the morning around 7:00 to 8:00 AM. Based upon the data provided by this field test of an exemplary embodiment of the portable environment monitoring system 300, it was established that the user was experiencing higher than normal VOC exposure when returning home at night and sustaining that exposure until leaving the home in the morning. Further analysis of the user's home in the particular field test resulted in the discovery of an open gas source in the user's garage. This open gas source was resulting in a higher than normal VOC level concentration not only in the user's garage, but also in the entire home of the user.

While the invention has been disclosed in its preferred forms, it will be apparent to those skilled in the art that many modifications, additions, and deletions can be made therein without departing from the spirit and scope of the invention and its equivalents as set forth in the following claims.

1. A portable environment monitoring system comprising:
a sensor enabled to sense an airborne analyte;
a respiratory monitoring device;
a microprocessor in communication with the sensor and enabled to process information received from the sensor and information received from the respiratory monitoring device;
a memory device in communication with the microprocessor and enabled to store information received from the microprocessor; and
wherein a user is enabled to ambulate with the system.

2. (canceled)

3. The portable environment monitoring system of claim 1, wherein the respiratory monitoring device is enabled to provide data regarding a pulmonary function of the user.

4. The portable environment monitoring system of claim 3, wherein the respiratory monitoring device is enabled to identify a reduction in the pulmonary function of the user.

5. The portable environment monitoring system of claim 1, wherein the sensor, the microprocessor, and the memory device are contained within a housing and, together with the housing, have a total specific weight of less than five pounds.

6. (canceled)

7. The portable environment monitoring system of claim 6, wherein the housing containing the sensor, the microprocessor, and the memory device has a specific total weight of less than one pound.

8. The portable environment monitoring system of claim 1, wherein the sensor is a volatile organic compound sensor.

9. The portable environment monitoring system of claim 8, further comprising a carbon dioxide sensor, nitrogen dioxide sensor, an ozone sensor, and a formaldehyde sensor.

10. A method for determining a respiratory deficiency trigger comprising:
providing a user with a portable environment monitoring system comprising a sensor, a microprocessor, a memory device, and a respiratory monitoring device;
collecting a plurality of data from the sensor and the respiratory monitoring device with the user in a plurality of environments;
analyzing the plurality of data received from the sensor and the respiratory monitoring device; and
determining whether a relationship exists between the exposure of the user to an airborne analyte and a deficient pulmonary function by the user.

11. The method for determining an asthmatic trigger of claim 10, wherein the user is enabled to ambulate with the portable environment monitoring system.

12. The method for determining an asthmatic trigger of claim 10, wherein the deficient pulmonary function by the user corresponds to asthmatic reaction.

13. The method for determining an asthmatic trigger of claim 12, further comprising establishing an asthmatic trigger based on the determination of the relationship between the exposure of the user to the airborne analyte and the deficient pulmonary function by the user.

14. The method for determining an asthmatic trigger of claim 10, wherein the collecting of a plurality of data from the sensor is automatically performed by the portable environment monitoring system.

15. The method for determining an asthmatic trigger of claim 14, wherein the collecting of the plurality of data from the sensor is done at regular intervals.

16. The method for determining an asthmatic trigger of claim 15, wherein the analyzing of the plurality of data from the sensor is executed by an external computer independent of the portable environment monitoring system.

17. A portable environment monitoring system comprising:
a garment enabled to be worn by a user comprising,
a sensor enabled to sense an airborne analyte;
a respiratory monitoring device;
a microprocessor in communication with the sensor and enabled to process information received from the sensor and information received from the respiratory monitoring device;
a memory device in communication with the microprocessor and enabled to store information received from the microprocessor; and
wherein a user is enabled to ambulate while wearing the garment.

18. (canceled)

19. The portable environment monitoring system of claim 17, wherein the respiratory monitoring device is enabled to provide data regarding a pulmonary function of the user.

20. The portable environment monitoring system of claim 17, wherein the sensor is a volatile organic compound sensor.

21. The portable environment monitoring system of claim 20, further comprising a carbon dioxide sensor, nitrogen dioxide sensor, an ozone sensor, and a formaldehyde sensor.

22. The portable environment monitoring system of claim 17, wherein the sensors, the microprocessor, and the memory device are contained within a housing.

23. The portable environment monitoring system of claim 22, wherein the housing containing the sensors, the microprocessor, and the memory device has a specific total weight of less than two pounds.
